(19) United States
(12) Patent Application Publication
(10) Pub. No.: US 2009/0306600 A1
(43) Pub. Date: Dec. 10, 2009

(54) Gastrostomy Tube, Production Methods Thereof and Insertion Tool

(76) Inventor: Stephane Regnault, Rueil-Malmaison (FR)

Correspondence Address: LEVINE & MANDELBAUM, 222 Bloomingdale Road, Suite 203, WHITE PLAINS, NY, 10605, US

(21) Appl. No.: 12/306,354

(22) PCT Filed: Jul. 6, 2007
(86) PCT No.: PCT/EP2007/056912

§ 371 Date: Dec. 23, 2008

(30) Foreign Application Priority Data
Jul. 6, 2006 (FR) 06/06139

(51) Int. Cl. A61M 25/04; A61M 25/01; B29C 45/00

(52) U.S. Cl. 604/175; 606/108; 264/328.1

(57) ABSTRACT

The invention relates to a gastrostomy tube, to the production methods thereof and to an insertion tool. The distal end (2) of the tube (1) is provided with a bulb (10) formed by parallel strips (6) which are provided in an area of the wall of the tube at a distance from the tip (2) at the distal end, said strips being separated by slits (4) in the wall, but being joined at each end (6a, 6b) to the tube wall. The strips curve outwards to form the bulb with the tip of the tube retracted inside the bulb, such that said tip must be pushed back out of the bulb in order to for the strips to be stretched, thereby eliminating the bulb and enabling the tube to be pulled through the stoma. The invention is suitable for use in gastrostomy.

The invention concerns a gastrostomy tube at the distal end of which is a deformable bulb between a stable configuration referred to as a retained configuration in which this bulb is too large to pass through the passage (“stoma”) formed through the skin and the gastric wall and a retaining configuration provoked by an appropriate solicitation action in which this bulb is reduced small enough to pass through the passage while the bulb recovers elastically its stable configuration when the solicitation is eliminated, and the instrument for installing the gastrostomy tube.

Publication EP 0742 708 divulges an embodiment in which the distal end of the tube is opened, having been spread and curved, to form a hollow retaining bowl around the end of the tube, this end continuing to protrude ahead of the bulb.

Publication WO 95/18640 (=U.S. Pat. No. 5,356,391) describes an embodiment in which the distal end of the tube is open and has been spread curved to form a retaining bulb at the end of the tube, this end continuing to protrude ahead of the bulb.

In both cases, the material of the bulb is chosen so that the bulb can be formed in an elastic manner from one configuration to the other under the effect of the action applied to the bulb from the outside of the skin.

Accordingly, in the aforementioned examples, this deformation can be caused by pulling on the tube.

It sometimes happens that this pulling action is caused in an inopportune manner by the patient.

This invention aims at avoiding any such accidents.

This is achieved according to the invention with a gastrostomy tube at the distal end of which is a bulb designed to retain the tube in the gastric cavity and which is suitable to be reduced in an elastic manner on demand to allow the tube to be inserted or extracted through the stoma, characterised whereby the bulb comprises wall strips formed in an area of the tube wall at some distance from the end of the distal end, these strips being separated by slits in the wall of the tube but remaining adjacent to this wall at each end of the strip and in which the scripts are curved outwards to form the bulb and the distal end of the tube is retracted into the bulb so that it is necessary to push this end back out of the bulb for the strips to be extended and recover their original positions, eliminating the bulb and allowing the tube to be inserted or withdrawn through the stoma.

To push back the end of the tube, a mandrel is used, inserted on demand into the tube.

It has been proposed (U.S. Pat. No. 5,248,302) to use a mandrel to push back the head of a retaining part fitted to the end of the gastrostomy tube in order to extend the part and allow it to pass through the stoma but the shape of the retaining part did not prevent the same results being obtained by a pulling effect on the tube.

In the following, we describe a typical embodiment of a gastrostomy tube conforming to the invention with reference to the figures of the attached illustrations and in which:

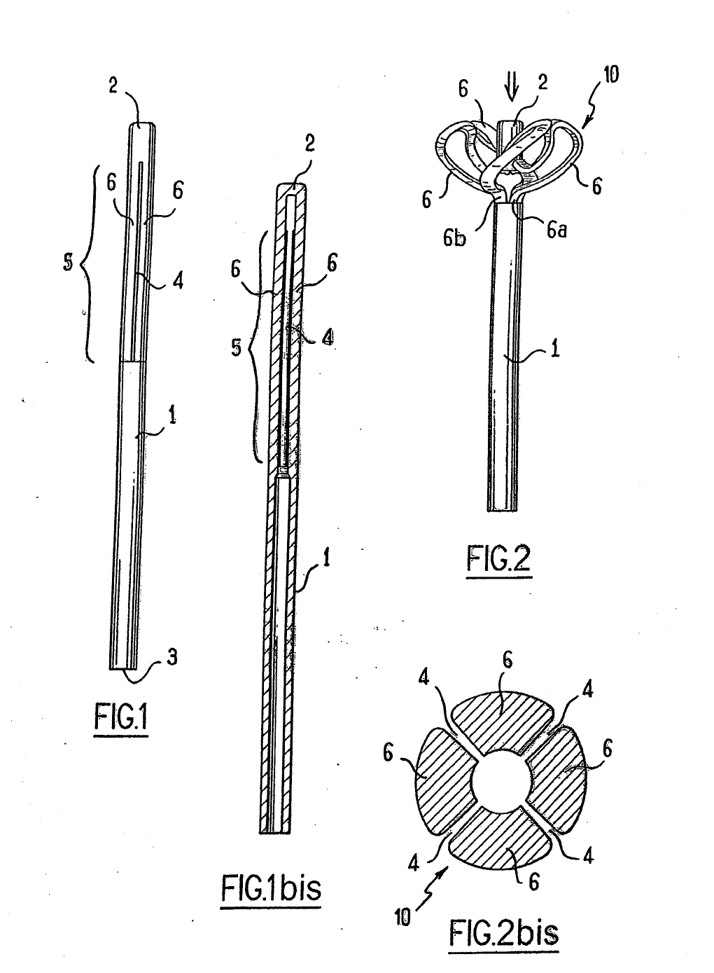

FIG. 1 is a longitudinal diagram of an example of the embodiment of a gastrostomy tube conforming to the invention, in the course of a phase preparatory to the forming of a bulb at one end of the tube;

FIG. 1 bis is an axial section of the tube in FIG. 1;

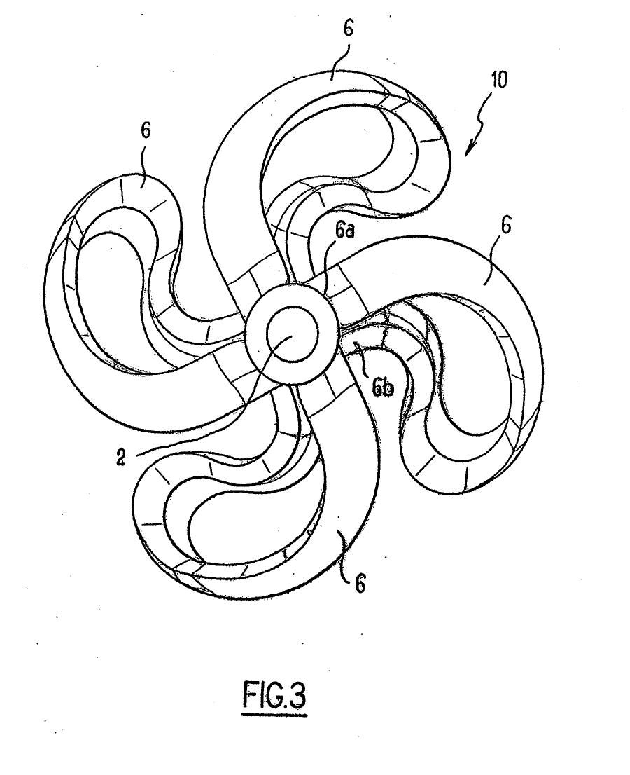

FIG. 2 is a similar diagram in the course of the forming of the bulb;

FIG. 2 bis is a horizontal section of the bulb;

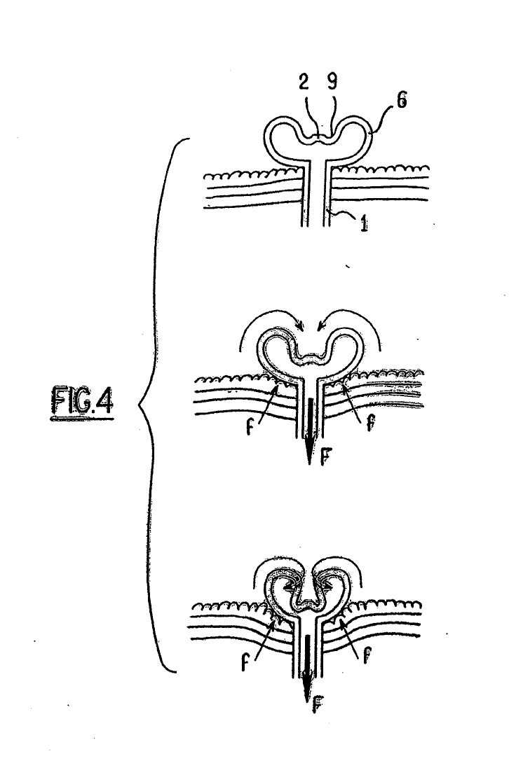

FIG. 3 is an end view of the formed bulb;

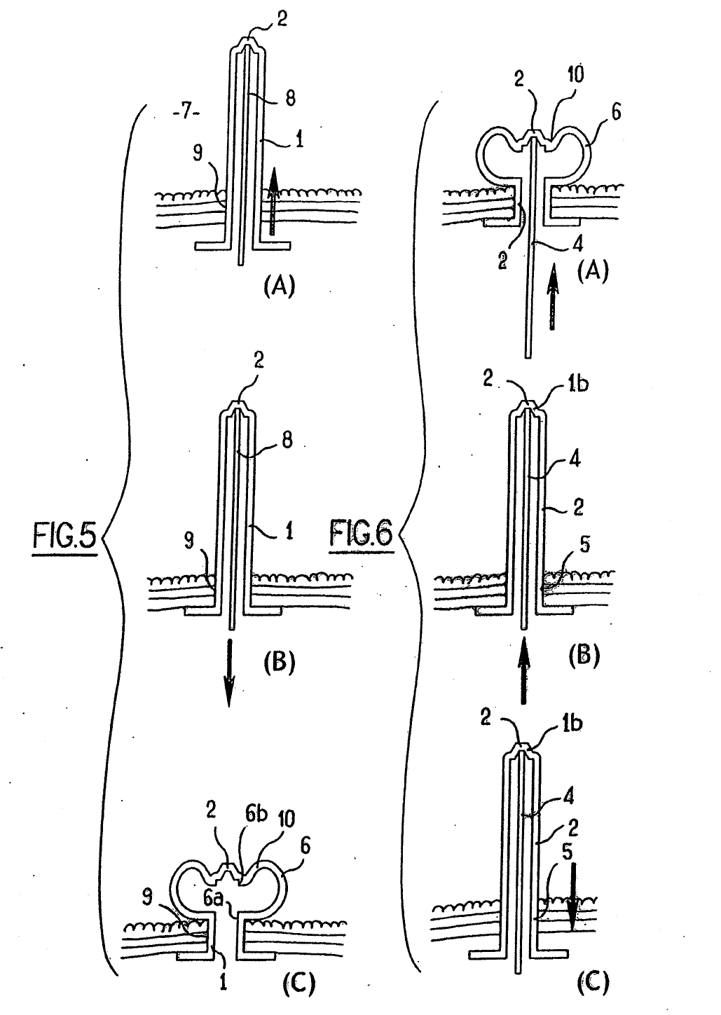

FIG. 4 represents diagrams of the bulb in a vertical section during the various pulling phases applied to the bulb;

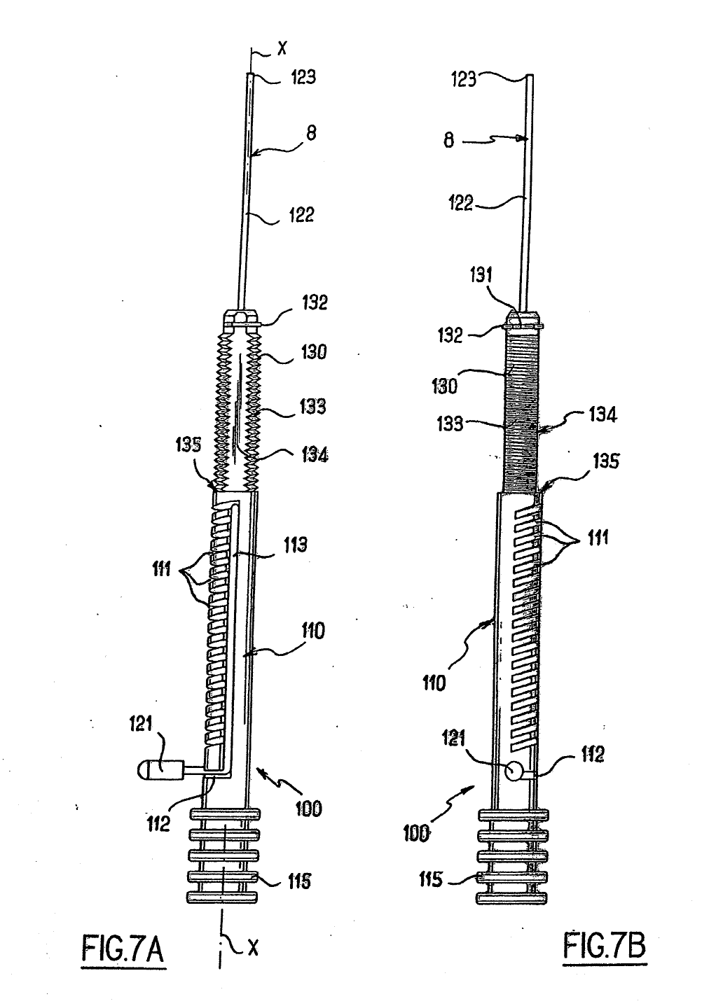

FIG. 5 represents diagrams of the tube in the course of the insertion of the tube into the stoma;

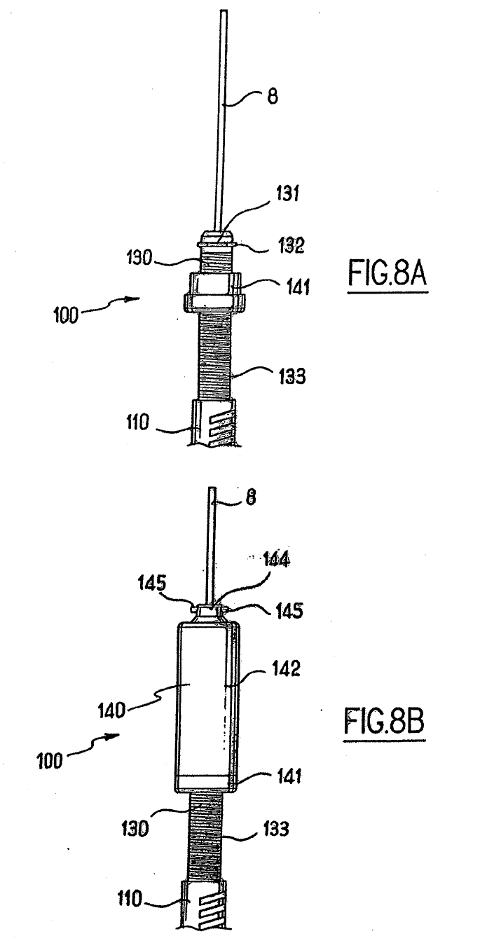

FIG. 6 represents diagrams of the tube in the course of the extraction of the tube out of the stoma;

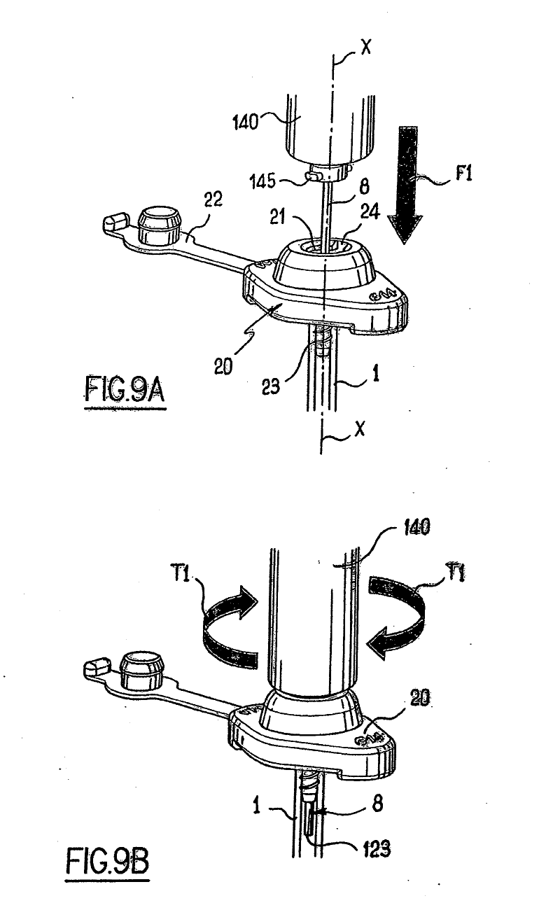

FIGS. 7a and 7b are front and profile views of an instrument for installing a gastrostomy tube according to the invention, which instrument does not have a handle;

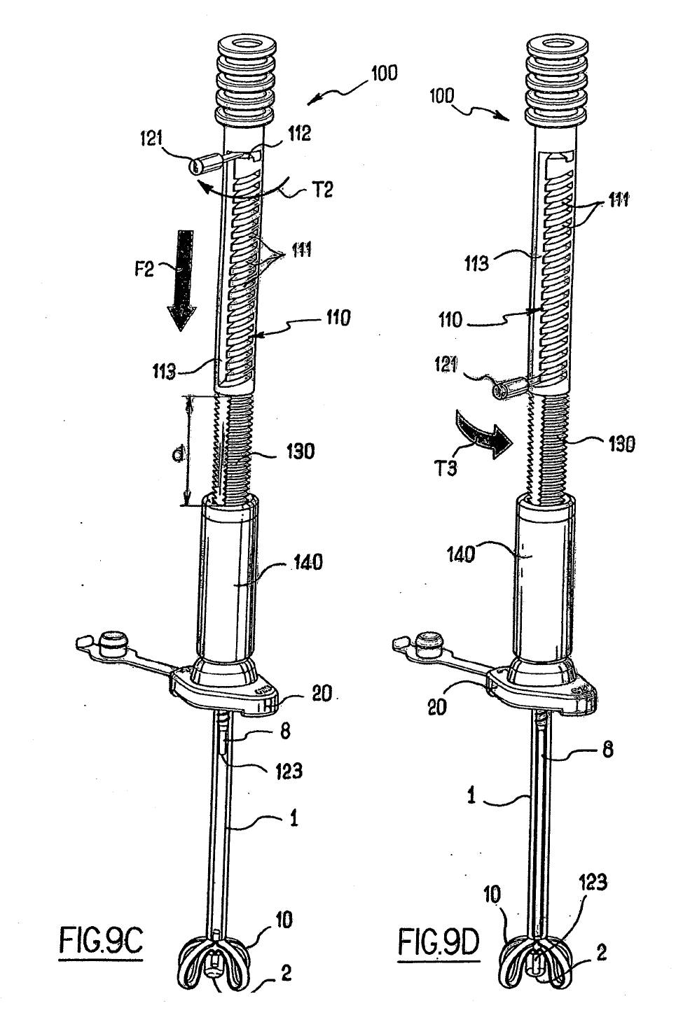

FIGS. 8a and 8b are a partial profile view of the distal end of the instrument for installing a gastrostomy tube of FIGS. 7a and 7b showing the assembly of the handle; and

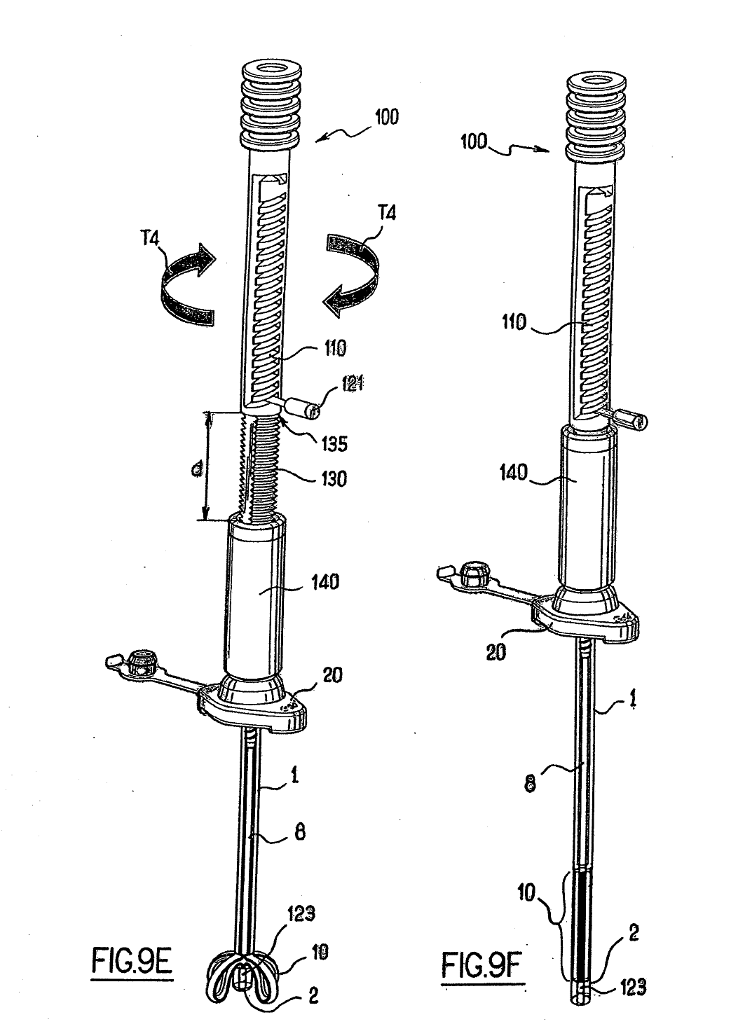

FIGS. 9a to 9f are three-dimensional views of the various stages of the use of the instrument to install the gastrostomy tube of FIGS. 7a to 7b with a gastrostomy tube according to the invention.

The gastrostomy tube shown in the figures is a cylindrical rectilinear tube (1) at the distal end of which is a closed end (2). In this example, the end is entirely closed but in an alternative, it could have holes in it as long as they do not compromise the possibility of pushing the end by means of a mandrel inserted from the other (open) end (3) of the tube.

In conformity with the invention, slits (4), (FIG. 1), having been made in a zone (5) of the tube wall, at a distance but preferably not far from the closed end (2) to determine in the wall of the tube the wall strips with each strip (6) being determined by two successive slits and remaining adjacent to the tube at either end (6a, 6b) of the strip.

Slits (4) can be arranged in parallel to the axis of the tube, the simplest case, or may be at an oblique angle with respect to this axis.

The slits can be obtained by cutting or by moulding.

Closed end (2) of the tube is then pushed back end-wise towards open end (3) of the tube, forcing strips (6) to curve towards the outside of the tube while forming a bulb (10) within which is closed end (2) which was pushed back (FIGS. 2 and 3).

In the process of crushing, the end of the tube turns about itself so that the strips forming the bulb are twisted to some extent.

To freeze the shape of the bulb, the end of the tube is heated.

The ready-to-use tube therefore has a bulb at its closed end. To insert the tube through a stoma (9) as far as the gastric cavity (7), it is extended by means of a mandrel (8) inserted into the tube to push back the closed end (2) and bring the strips into the wall of the tube (FIG. 5A).

Then, the mandrel is withdrawn (FIG. 5B) and the bulb recovers its initial configuration in an elastic manner (FIG. 5C).

It will be seen in FIG. 5C that each strip (6), before connecting to the tube near the closed end (2) has an inflection (10) forming a preliminary curve opposing the extension so that any force applied to the tube (FIG. 4) would cause the bulb to contract without being reduced sufficiently to allow the tube to be extracted through the stoma.

For extraction to be possible, it is necessary to reinsert the mandrel into the tube to push back the closed end and extend the tube until the bulb is suppressed (FIG. 6).

In FIGS. 4 to 5, the tube is represented in a simplified manner, in particular concerning its open end (3) which will remain outside the skin in a known embodiment.

The number of strips (6) arranged as if they were the petals of the bulb is chosen to order and could be, for instance, four.

The invention is not limited to the embodiment described here.

Preferably, a polyurethane or similar tube will be used.

Polyurethane is a thermoplastic material injected into a mould for extrusion at temperatures ranging between 170 and 200° C. It is also possible to hot-shape polyurethane in an oven at 150° C.

In the stomach, and at ambient temperature, it keeps its shape and is elastic. When it is deformed, it recovers its shape as soon as the force stops.

Polyurethane material is the best suited to this use because of its resistance to the gastric fluids and to bacteria (candida albicans), its biocompatibility and its elasticity.

With reference to FIGS. 7a to 7b, we will describe an instrument (100) for installing a gastrostomy tube according to the invention.

The insertion tool (100) has a body (110) and a mandrel (8).

Mandrel (8) has a stem (122) extending according to a longitudinal axis X of the installing instrument (100), a distal end (123) and a proximal end bent at approximately 90° including a handle (121).

Body (110) is cylindrical in a form that could be a revolution about a longitudinal axis X. At a proximal end it has a handle (115). In addition, at a distal end (130), body (110) has threading (133) extending over some length in the direction of longitudinal axis X as well as two flats (134) arranged diametrically opposite one another on a longitudinal axis X.

In addition, distal end (130) of body (110) includes a circumferential groove (131) arranged more or less perpendicular to axis X, which is capable of receiving a circlip (132). Threading (133) extends distally approximately from groove (131) to proximally an edge (135) forming a stop. Between handle (115) and threading (133), body (110) has a slot (112) more or less perpendicular to longitudinal axis X and which extends over approximately 90° on the circumference of body (110) as well as a series of slits (111) inclined with respect to longitudinal axis X and more or less parallel to one another. This series of slits (111) in the same way as slit (112) extend over approximately 90° on the circumference of body (110). In addition, body (110) has a longitudinal slit (113) more or less parallel to longitudinal axis X, on a generating line of body (110) connecting among other things the ends on the same side of slits (112) and (111). In addition, body (110) has a longitudinal hole on axis X extending from distal end (130) to at least one slit (112).

During the assembly of the insertion tool (100), distal end (123) of stem (122) of mandrel (8) is inserted into the longitudinal hole on axis X through threads (113) of body (110). Stem (122) is slipped into this longitudinal hole in axis X until the bent proximal end of mandrel (8) can be inserted into slit (112) of body (110). A nut (141) is screwed onto distal end (130) so that it meshes with threads (133) of body (110). Then, circlip (132) is fitted into groove (131) of distal end (130) of body (110).

This circlip (132) serves as a stop for nut (141) when the latter is screwed in or screwed out towards distal end of body (110). A sleeve (142) is tight-fit on nut (141) to form an assembly (140). In an alternative embodiment, a sleeve (142) is screwed onto nut (141) or welded or bonded. Sleeve (142) has a distal end (144), two diametrically opposed bayonets (145) with respect to one another, along the longitudinal axis X of insertion tool (100).

With reference to FIGS. 9a to 9f, we will describe the use of insertion tool (100).

Prior to using insertion tool (100), gastrostomy tube (1) according to the invention is provided with a base (20) having means of assembly (23) on gastrostomy tube (1) located on the lower face of base (20). On an upper face of base (20), the latter has an opening (21) on the perimeter of which are located, diametrically opposed with respect to longitudinal axis X, notches (24), of which there are two in this case. Finally, base (20) has a plug (32) suitable to be inserted into hole (21) to close the gastrostomy tube in a sealed manner. Fuller information concerning base (20) and its assembly on gastrostomy tube (1) are given in the request for French patent number FR 060 6140 which can be referred to.

Initially, assembly (140) formed by sleeve (142) and nut (141) is screwed onto body (110) of insertion tool (100) towards a distal end until nut (141) abuts against circlip (132). The proximal bent end of mandrel (8) is placed in slot (112) of body (110) of insertion tool (100) while the distal end (123) of mandrel (8) protrudes from the distal end of sleeve (142). Then, distal end (123) of mandrel (8) is inserted into gastrostomy tube (1) through a hole (21) of base (20) along the longitudinal axis X in the direction of arrow F1. A movement according to arrow F1 is made until the distal end (144) of sleeve (142) is inserted into a hole (21) of base (20) with bayonets (145) passing through notches (24). Sleeve (140) is then locked in base (20) during a rotation movement T1 about axis X in order to render integral base (20) and insertion tool (100) by a “bayonet” assembly as illustrated in FIG. 9b.

Then, by a rotation movement T2, the bent proximal end of mandrel (8) is extracted from slit (112) as far as longitudinal slit (113). Mandrel (8) is then able to slide according to a movement F2 as its proximal bent end moves inside longitudinal slit (113). Movement F2 is made slightly until distal end (123) of mandrel (8) comes into contact with closed end (2) of gastrostomy tube (1), bulb (10) of gastrostomy tube (1) then being formed at rest. Once distal end (123) of mandrel (8) bears in contact with closed end (2), bent proximal end of mandrel (8) is then inserted into one of the slits (111) which are then found facing each other, in a rotation movement T3. From that moment on, body (110) is put into a rotation movement according to T4 which results in screwing body (110) into assembly (140) by the cooperation of nut (141) assembly (140) and threading (133) of distal end (130) of body (110) of insertion tool (100). This screwing movement takes place over a predetermined and limited distance (d). Once distance (d) is covered, nut (141) of assembly (140) abuts at one proximal end of threading (133) on edge (135), as illustrated in FIG. 9f. During this screwing in movement, distal end (123) of mandrel (8) pushes closed end (2) of gastrostomy tube (1) out of bulb (10) until the latter disappears so that the strips forming bulb (10) are completely extended and distance (d) is covered. Then, gastrostomy tube (1) can be removed from the patient or inserted into the patient through the stoma.

Insertion tool (100) described above and its use avoid having to push by hand and directly on the mandrel to extend the strips forming the bulb and thus avoid any risk of perforating the opposite wall of the stomach if the mandrel passes through or escapes from the closed end of the gastrostomy tube. In addition, thanks to the series of slits (111), insertion tool (100) can be adjusted according to the length of the gastrostomy tube with which it is designed to work, and this insertion tool subsequently allows a thrusting travel which is always identical as needed to extend the strips forming the bulb. The controlled achieving of this travel ensures the user that the gastrostomy tube is ready to be installed or removed. This is particularly important when removing the gastrostomy tube, because the user does not see the state of deformation of the bulb in the stomach before withdrawal. Accordingly, insertion tool (100) according to the invention allows manipulation of a gastrostomy tube according to the invention with optimal safety.

There is no limit on the invention either to the embodiment described or to the material which is recommended.

1. Gastrostomy tube with a distal end having a bulb for retaining the tube in a gastric cavity and suitable for elastic reduction on demand to allow the introduction or extraction of the tube through the stoma, wherein said bulb comprises strips in an area of a tube wall located at a distance from said distal end, such strips being separated by slits in the wall but remaining adjacent to the wall of the tube at either end of the strip and whereby the strips are curved inwards towards the outside to form said bulb and in that said end of the tube is withdrawn into the bulb so that it is necessary to push the end of the tube out of the bulb for the strips to be extended, thus eliminating the bulb and allowing the tube to be inserted or withdrawn via the stoma.

2. A tube according to claim 1 in which said strips forming the bulb are twisted.

3. A tube according to claim 1 in which each strip, before being connected to the tube near said distal end has an inflection forming a preliminary curve opposing the extension so that any force applied to the tube causes the bulb to contract without being able to be reduced sufficiently to allow the extraction of the tube through the stoma.

4. A tube according to claim 1 in which said slits in the tube wall are parallel to the tube wall.

5. A tube according to claim 1 wherein said end is closed.

6. A tube according to claim 1 the material of which comprises polyurethane.

7. A process for manufacturing a tube comprising splitting a closed tube at one end to form, in a wall of the tube, wall strips, crushing the closed end of the tube to push the strips outward and form a bulb, and heating the tube to stabilise the bulb.

8. A process of manufacturing a tube comprising molding the tube by the injection of polyurethane plastic or similar material into a mold forming the tube, its closed end and said slits, crushing the end of the tube to form a bulb, and heating the tube to stabilise the bulb.

9. Insertion tool for a gastrostomy tube comprising a mandrel having a distal end designed to come to bear against an end of said gastrostomy tube, means for gradually driving said mandrel over a limited predetermined distance so that, during implementation, said distal end of said mandrel pushes said end of said tube out of a bulb so that strips are drawn out.

10. Insertion tool according to claim 9 wherein the driving means include a nut meshing with threads for screwing said mandrel in and out.

11. Insertion tool according to claim 9 wherein the mandrel has a bent proximal end and said driving means has a plurality of slits suitable for receiving the bent proximal end.
